(19) United States
(12) Patent Application Publication
(10) Pub. No.: US 2010/0280047 A1
(43) Pub. Date: Nov. 4, 2010

(54) SALTS OF ACTIVE INGREDIENTS WITH POLYMERIC COUNTER-IONS

(75) Inventors: Karl Kolter, Limburgerhof (DE); Maximilian Angel, Schifferstadt (DE); Kathrin Meyer-Böhm, Feucht (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/747,557

(22) PCT Filed: Dec. 10, 2008
(86) PCT No.: PCT/EP08/67228

§ 371 Date: Jun. 11, 2010

(30) Foreign Application Priority Data
Dec. 12, 2007 (EP) 07122995.9

(51) Int. Cl. A61K 31/495; A61K 31/45; A61K 31/426; A61P 1/04; A61P 1/08; A61P 25/18; A61P 1/12

(52) U.S. Cl. 514/255.04; 514/327; 514/370

(57) ABSTRACT

Polymeric water-soluble salts of medicaments that are sparingly soluble in water, consisting of a polymer with anion character that is soluble in water at pH values of 2-13 and a sparingly soluble medicament with cation character.

The present invention relates to solid salts of active ingredients with polymeric counterions.

Numerous medicaments have a very low solubility in water and can consequently not be absorbed from the gastric and intestinal tract. The result is very low bioavailability. For medicaments which have a basic or acidic group, corresponding salts can be formed through reaction with acids or alkalis, said salts sometimes having better solubilities. For this purpose, low molecular weight acids or alkalis are usually used. The most common acids are: hydrochloric acid, sulfuric acid, acetic acid, citric acid, tartaric acid, fumaric acid, maleic acid, malonic acid, succinic acid, phosphoric acid. The bases used are NaOH, KOH, L-lysine, L-arginine, triethanolamine or diethylamine. However, with many medicaments, the salts with these low molecular weight compounds are also difficulty soluble in water. Often, there is barely any difference between the solubility of the medicament acid or base and that of a salt with the specified compounds. The cause of this poor solubility is usually that the salt forms a very stable crystal lattice which is energetically in a favorable state, as a result of which the tendency to dissolve is low. If, additionally, the energy gain as a result of hydration is low, the solubility is further reduced.

Salts of medicaments with polymeric acids or bases have hitherto already been produced in principle, although polymers were used which were not soluble over a large pH range—in particular not in the physiologically relevant range of pH 2-8—or which in solution as acid, base or salt, have a high viscosity. If polymers are used which are insoluble at acidic pH values, as is the case with polymers that are resistant to gastric juice, no dissolution of the medicament takes place, but there is precipitation of the polymer. As a result, the release is prevented or at least greatly slowed. This results in a preparation that is resistant to gastric juice which lowers the bioavailability since, on the one hand, no absorption can take place in the stomach and, on the other hand, dissolution of the preparation must only take place in the small intestine at neutral pH values, as a result of which release takes place relatively late and the entire small intestine surface is no longer available for the absorption. If the polymers have high viscosity in aqueous solution, the active ingredient release from a solid administration form such as, for example, a tablet, is likewise delayed.

Upon dissolution of the salt, a gel or a highly viscous solution forms on the surface of the tablet and in the cavities, preventing further penetration of the water into the tablet core and slowing disintegration. This effect and also the reduced diffusion coefficients of the medicament molecules through areas with high viscosity delay the release of the medicament. This option of delayed release is utilized when producing matrix slow-release forms with polymers of high viscosity such as, for example, alginates, xanthans, methylhydroxymethylcelluloses, sodium carboxymethylcelluloses, pectins etc. However, these polymers are in no way suitable for producing rapid-release forms in which a sparingly soluble medicament is to be dissolved quickly and provided to the entire gastric and small intestine surface for absorption.

EP 0211268 describes minoxidil salts with polymeric polyanions which have delayed release and are used for dermal application. Minoxidil is a medicament which comprises 4 groups capable of salt formation and the corresponding polymeric salts were less soluble than the hydrochloride. The numerous groups capable of salt formation greatly reduce the dissociation of the salt and does not improve the solubility compared with the hydrochloride. Oral applications are not described.

U.S. Pat. No. 4,997,643 describes a biocompatible, film-forming delivery system for topical application which comprises a polymeric salt with a carboxyl-group-carrying component. The medicament used is likewise minoxidil, which has the aforementioned special characteristics. Oral applications are not described.

U.S. Pat. No. 4,248,855 claims liquid preparations which comprise salts of basic medicaments and water-insoluble polymers, and which have a slow-release effect. As a result of the use of water-insoluble polymers, the preparations do not exhibit rapid release or high solubility over a large pH range.

U.S. Pat. No. 5,736,127 discloses that salts can be formed from basic medicaments and polymers with carboxyl-amidine-carboxyl triads. On account of the high molecular weights, the polymers are gel-forming, as a result of which the release of the active ingredients is delayed. Suitability for rapid-release tablets is not stated.

U.S. Pat. No. 4,205,060 describes microcapsules with delayed release which comprise, in the core, a salt of a basic medicament with a carboxyl-group-containing polymer and which is surrounded by a water-insoluble polymer. The carboxyl-group-containing polymer reduces the release of the soluble medicaments used.

Salts of ranitidine with polycarboxylic acids are described in EP 0721785. The polycarboxylic acids bind the ranitidine and reportedly reduce its bitterness. However, low molecular weight salts of ranitidine are readily soluble, meaning that the polycarboxylic acids merely restrict the mobility and diffusion of the ranitidine, meaning that it does not pass so rapidly to the bitter receptors.

It was an object of the present invention to find active ingredient salts which, following processing to give oral dosage forms, permit a more rapid release of the active ingredient compared to the corresponding hydrochloride salt. Furthermore, the sought active ingredient salts should also be soluble irrespective of the pH. It was furthermore an object to find those salts which permit good tabletability of the active ingredient.

Accordingly, water-soluble salts of medicaments with polymeric counterions have been, consisting of a polymer with anion character that is soluble in water at pH values of 2-13 and a medicament with cation character that is sparingly soluble in water, where the polymer with anion character used is a polymer which has a fraction of at least 40% by weight of carboxyl-group-carrying monomers.

Furthermore, methods for their production and processing to give solid administration forms have been found.

The polymers with anion character used are non-gel-forming polymers.

The polymer with anion character preferably has a fraction of at least 50% by weight of acrylic acid and/or methacrylic acid.

According to the invention, active ingredient bases are referred to as active ingredients with cation character. Preferred active ingredients with cation character are those with at least one and at most two groups capable of salt formation.

The respective polymeric counterions are selected such that the polymeric medicament salt has a higher solubility in water than the medicament and the corresponding hydrochloride of the medicament.

As a result of the described salt formation, it is also possible to dissolve medicaments for which neither the neutral form nor the corresponding low molecular weight salts are soluble in water. For these medicaments, the dissolution in the gastric and intestinal tract is very slow and thus limiting for the absorption, as a result of which low bioavailability often results (according to the Biopharmaceutical Classification System: active ingredients of class II (Amidon et al., Pharm. Res. 12, 413-420)). The invention is applicable in particular to medicaments which, in uncharged form or as hydrochloride, have solubilities of less than 0.1% (m/m) in water, artificial intestinal juice or gastric juice.

A prerequisite for a considerably increased solubility over a broad pH range is that the polymers used are likewise water-soluble over a broad pH range. Only then do changes in the pH not result in polymer precipitations. The polymers should also not be gel-forming.

In order not to hinder the dissolution of the active ingredient from the polymeric salt, the viscosity of the polymer used should be low. Polymers which, in 5% strength by weight solution, have viscosities of less than 100 mPas, preferably of less than 75 mPas and particularly preferably of less than 50 mPas, have proven advantageous. Low viscosities as a rule require low molecular weights. Preferably, uncrosslinked polymers are used. For practical reasons, instead of the molecular weight, the K value is often determined. Here, the polymers have a K value of 150, preferably <90, particularly preferably <50, in particular <40 (measured at 1% strength by weight in water).

The glass transition temperature of the polymers should be above 60° C. so that the polymeric salts can be readily processed to give tablets and no changes arise in the stability test at slightly elevated temperatures.

The polymers with anion character can be homopolymers, copolymers, block copolymers, grafted copolymers, comb copolymers or star copolymers.

To produce the polymers with anion character, monomers which carry acidic groups, such as carboxyl groups, sulfonic acid groups, phosphoric acid groups or phosphonic acid groups (also called “acidic monomers” below), can be used.

The fraction of acidic monomers is preferably more than 80% by weight and particularly preferably more than 90% by weight, in particular more than 98% by weight. Here, the fraction of carboxyl-group-carrying monomers of the total amount of all monomers is at least 40% by weight, of which preferably at least 50% by weight are acrylic acid and/or methacrylic acid.

Monomers of these groups which are suitable in principle are:

monomers comprising sulfonic acid groups, such as vinylsulfonic acid, methylallylsulfonic acid, styrene sulfonic acid, toluene sulfonic acid, 2-sulfoethyl methacrylate, 2-acrylamido-2-methylpropanesulfonic acid, 3-sulfoethylpropyl acrylate or allylsulfonic acid,
monomers carrying phosphonic acid or phosphoric acid groups, such as vinylphosphonic acid, vinyldiphosphonic acid, 2-(methacryloyloxy)ethylphosphoric acid, 2-(acryloyloxy)ethylphosphoric acid,
carboxyl-group-carrying monomers, such as acrylic acid, methacrylic acid, maleic acid, maleic anhydride, crotonic acid, 4-oxo-2-pentenoic acid, monovinyl 1,6-hexane-dicarboxylate, hydroxy[(1-oxo-2-propenyl)amino]acetic acid, mono[2-[(2-methyl-1-oxo-2-propenyl)oxy]ethyl]1,4-butanedicarboxylate, 2-carboxyethyl acrylate, monovinyl 1,4-butanedicarboxylate, 6-[(2-methyl-1-oxo-2-propenyl)amino]hexanoic acid, 2-carboxypropyl methacrylate, 2-methyl-3-[(2-methyl-1-oxo-2-propenyl)amino]-1-propanesulfonic acid, 2-methyl-2-pentenoic acid.

Two or more acidic monomers can also be combined with one another.

Preferred sulfonic acid monomers are 2-sulfoethyl methacrylate, 3-sulfopropyl acrylate, 2-acrylamido-2-methylpropanesulfonic acid and allylsulfonic acid. Particularly preferred carboxyl-group-carrying monomers are acrylic acid, methacrylic acid, maleic acid or maleic anhydride.

Besides the homopolymers of acrylic acid, particular preference is given to the copolymers of acrylic acid and maleic acid and also copolymers of acrylic acid and methacrylic acid or copolymers of acrylic acid or methacrylic acid with itaconic acid. Itaconic acid can here be copolymerized in amounts of from 1 to 20% by weight, preferably 3 to 15% by weight, based on the total weight of the monomers.

Particular preference is also given to copolymers of acrylic acid or methacrylic acid with sulfonic acid monomers, in particular copolymers of acrylic acid or of methacrylic acid with 2-acrylamido-2-methylpropanesulfonic acid or 2-sulfoethyl methacrylate. Such copolymers can comprise 1 to 20, preferably 2 to 15, particularly preferably 5 to 10, % by weight of sulfonic acid monomers.

Said acidic monomers can be polymerized together with neutral comonomers. The neutral monomers can be water-soluble or water-insoluble. In the case of insolubility in water, the monomer fraction is limited by the fact that the polymer becomes water-insoluble at some point.

Suitable neutral comonomers are:

ethylene, propylene, butylene, styrene, methyl vinyl ether, esters of acrylic acid with C1-C20 alcohols, esters of methacrylic acid with C1-C20 alcohols, vinyl esters of C1-C20 carboxylic acids.

Furthermore suitable are hydroxyethyl acrylate, hydroxyethyl methacrylate, hydroxypropyl acrylate, hydroxypropyl methacrylate, (meth)acrylates with 5 to 50 ethylene oxide units, in particular the corresponding terminally capped compounds (e.g. methyl end group).

Copolymers of acidic and neutral monomers which comprise, as neutral monomers, monomers with substituted amide groups such as, for example, N,N-dimethylvinyl-formamide, N,N-dimethylvinylacetamide, N,N-dimethylacrylamide, N,N-diethylacryl-amide, N,N-dimethylmethacrylamide, diethylmethacrylamide, are particularly suitable. Very particularly suitable comonomers with substituted amide groups are the N-vinylamides such as N-vinylpyrrolidone, vinylcaprolactam, acrylamide, methacrylamide, in particular acrylamide.

The fraction of neutral monomers can be 1 to 60% by weight, preferably 1 to 20, particularly preferably 3 to 15% by weight, based on the total amount of the monomers.

Particular preference is also given to copolymers of two different acidic monomers and additionally neutral monomers, in particular copolymers of acrylic acid or of methacrylic acid with sulfonic acid monomers and N-vinylamide monomers.

The polymers according to the invention can be prepared in a manner customary per se by free-radical polymerization. Preferably, the polymerization takes place as solution polymerization in water. Such processes are known per se to the person skilled in the art. Suitable initiators are, for example, iron(II) sulfate, sodium peroxodisulfate or hydrogen peroxide.

It may also be advisable to carry out the polymerization in the presence of regulators, for example of sodium hypophosphite.

The polymerization can take place continuously or as a batch process, the polymers preferably being obtained via feed processes.

The conversion of the polymer solutions to the solid form can likewise take place by customary processes by spray-drying, freeze-drying or drum-drying.

During the production of the polymers according to the invention, it is preferably to be ensured that these have no low molecular weight anions such as, for example, chloride, sulfate, etc. which can lead to sparingly soluble salts with active ingredients.

However, from case to case, it may be advisable to use the polymeric salts according to the invention also in a mixture with sparingly soluble low molecular weight salts of the active ingredient or else in a mixture with the corresponding partially neutralized polymeric salts.

In principle, all pharmaceutical active ingredients, neutraceuticals, food additives and also crop protection active ingredients which have a basicity adequate for salt formation are suitable. For very weakly basic active ingredients, stronger acids such as sulfonic acids are thus suitable, for medium to strongly basic active ingredients it is also possible to use carboxylic acids or phosphonic acids. The pKa value of the acidic polymer must be equal to or less than the pKa value of the basic active ingredient. If this is not the case, then the salt is not stable against hydrolysis. It is particularly advantageous if there is a difference of at least 2 units between the pKa values of the polymers and of the active ingredient.

The production of the salts according to the invention can in principle take place by drying, melting or precipitation processes.

### Drying an Aqueous or Organic Solution

The active ingredient and the polymer are dissolved in water or organic solvents and the solution is then dried. The dissolution can also take place at elevated temperatures (30-200° C.) and under pressure.

In principle, all types of drying are possible, such as, for example, spray-drying, fluidized-bed drying, drum drying, freeze-drying, vacuum drying, belt drying, roller drying, carrier-gas drying, evaporation etc.

### Melting Processes

The active ingredient is mixed with the polymer. By heating to temperatures of 50-250° C., the production of the polymeric salt takes place. Here, temperatures above the glass transition temperature of the polymer or the melting point of the active ingredient are advantageous. By adding a softening auxiliary, such as, for example, water, organic solvent, customary organic softeners, it is possible to correspondingly reduce the processing temperature. Of particular advantage here are auxiliaries which can afterwards be very easily evaporated off again, i.e. having a boiling point below 180° C., preferably below 150° C.

Preferably, this type of production can be undertaken in an extruder.

The extruder is preferably a synchronous twin-screw extruder. The screw configuration can be shearing to a varying degree depending on the product. It is recommended to use kneading elements particularly in the melting zone. In this connection, it is also possible to use reverse kneading elements. The melting zone can be followed by a degassing zone, which is advantageously operated at atmospheric pressure, but if desired can also be operated under reduced pressure.

The product discharge can take place via round dies with diameters of from 1 to 5, preferably 2 to 3 mm. The extruders can also be equipped with die plates. Other die shapes, such as slit dies, can likewise be used, particularly if a relatively large material throughput is desired.

The extruders are usually equipped with heatable cylinders. The resulting product temperature, however, is dependent on the degree of shear of the screw element used and can sometimes be 20-30° C. higher than the established cylinder temperature.

Usually, extruders with a length of from 25D to 40D are suitable.

In principle, a plurality of active ingredient bases can also be reacted with the polymer in the extruder to give the salt.

The extrudate strands emerging from the extruder can be comminuted in a manner known per se, for example by chopping techniques.

The resulting strands can be processed using a granulator to give granules and these can in turn be further comminuted (ground) to give a powder. The granules or powder can be poured into capsules or be compressed to give tablets using customary tableting auxiliaries.

In one particular embodiment of the extrusion, the polymer can firstly be fed to the extruder and be melted. The active ingredient base is then added via a second entry point. Additional active ingredient bases can be metered in via further entry points.

It is furthermore possible to use water, organic solvents, buffer substances or plasticizers during the extrusion in order to increase the rate of the reaction of the active ingredient base with the acidic polymer. In particular water or volatile alcohols are suitable for this purpose. This process allows the reaction at a relatively low temperature. The amounts of solvent and/or plasticizer are usually between 0 and 30% of the extrudable mass. The water or solvent can be removed simply through a degassing point in the extruder at atmospheric pressure or by applying a vacuum. Alternatively, these components evaporate when the extrudate leaves the extruder and the pressure drops to atmospheric pressure. In the case of more difficulty volatile components, the extrudate can be afterdried as appropriate.

Two further embodiments consist in either dissolving the active ingredient in a solvent and feeding it into the polymer in the extruder, or dissolving the polymer in a solvent and feeding it into the active ingredient.

According to one embodiment of the preparation process, directly after the extrusion, the thermoplastic mass is calendered to give a tablet-like compact, which represents the ultimate administration form. In this variant, it may be useful to add further constituents such as, for example, polymers for adjusting the glass transition temperature and the melt viscosity, disintegrants, solubilizers, plasticizers, release-influencing auxiliaries, retardants, polymers which are resistant to gastric juice, dyes, flavorings, sweeteners and further additives either before or during the extrusion. In principle, these substances can also be used whenever the extrudate is firstly comminuted and then compressed to give tablets.

The water content of the extruded products is generally below 5% by weight.

The solubility in water of the polymers with anion character in the pH range 3-11 is greater than 10% (m/m).

By adding thermoplastic retarders such as polyvinyl acetate homopolymers or formulations of such polyvinyl acetate homopolymers, furthermore Eudragit® grades RS, RL or NE or NM known as retarder polymers based on acrylate, it is possible to slow the release. In this way, it is possible to produce slow-release forms of sparingly soluble medicaments which release in a reliable manner.

In principle, the reactions for the salt formation can also be carried out in a mixer as melt granulation or by wetting with water or a solvent as wet granulation. Granules in the wider sense can also be produced by means of wet extrusion with subsequent spheronization. The resulting pellets can then be poured into hard capsules as multiple dosage form.

### Precipitation Processes

The active ingredient and the polymer are dissolved in water or an organic solvent and then the temperature is rapidly reduced or a nonsolvent miscible with water or the organic solvent is added. This results in precipitation of the polymeric salt, which is then filtered off or centrifuged off and is dried. Suitable nonsolvents are, for example, acetone, isopropanol or n-butanol.

The described polymeric salts are always amorphous, thus avoiding the problem as in the case of the low molecular weight salts that the amorphous state can convert, as a result of external influences or in the course of storage, to a thermodynamically stable crystalline state. The amorphous state of the salts according to the invention is thus a thermodynamically stable state because there is no state lower in energy for such substances. The polymeric salts thus differ in principle from the low molecular weight salts.

Furthermore, with low molecular weight salts there is often the problem that they poorly crystallize and/or are formed with a low melting point. This problem is avoided with the described polymeric salts.

The polymeric salts according to the invention of a polymeric anion component and an active ingredient, which serves as basic cation component, have excellent processability to give administration forms, in particular with regard to tabletability. Consequently, it is possible to produce tablets with a diameter of 10 mm and 300 mg in weight which have a fracture resistance of more than 200 N. The polymeric anion therefore acts simultaneously as binder and imparts enormous plasticity to the tablet formulation. By contrast, low molecular weight salts are often very brittle and poorly tabletable.

The polymeric salts according to the invention are suitable for improving the solubility of all active ingredients which are poorly soluble, sparingly soluble or insoluble in water and which are able to form salts with the acidic groups of the polymer component. According to DAB 9 (German pharmacopeia), the classification of the solubility of pharmaceutical active ingredients is as follows: poorly soluble (soluble in 30 to 100 parts of solvent); sparingly soluble (soluble in 100 to 1000 parts of solvent); virtually insoluble (soluble in more than 10 000 parts of solvent). The ingredients can here come from any indication area.

Examples which may be mentioned here are antihypertensives, vitamins, cytostatics—in particular taxol, anesthetics, neuroleptics, antidepressants, antibiotics, antimycotics, fungicides, chemotherapeutics, urologics, platelet aggregation inhibitors, sulfonamides, spasmolytics, hormones, immunoglobulins, sera, thyroid therapeutics, psychoactive drugs, antiparkinson agents and other antihyperkinetics, opthalmologicals, neuropathy preparations, calcium metabolism regulators, muscle relaxants, narcotics, lipid-lowering agents, hepatotherapeutics, coronary agents, cardiac agents, immunotherapeutics, regulatory peptides and their inhibitors, hypnotics, sedatives, gynecologicals, gout remedies, fibrinolytics, enzyme preparations and transport proteins, enzyme inhibitors, emetics, slimming agents, blood flow stimulators, diuretics, diagnostic aids, corticoids, cholinergics, biliary therapeutics, antiasthmatics, bronchodilators, beta-receptor blockers, calcium antagonists, ACE inhibitors, arteriosclerosis remedies, antiphlogistics, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists or antiviral active ingredients or active ingredients for the treatment of HIV infections and AID syndrome.

During the preparation of the solid dosage forms according to the invention, customary pharmaceutical auxiliaries can if appropriate be co-processed. These are substances from the class of fillers, plasticizers, solubility promoters, binders, silicates and also disintegrants and adsorbents, lubricants, flow agents, dyes, stabilizers such as antioxidants, wetting agents, preservatives, mold release agents, aromas or sweeteners, preferably fillers, plasticizers and solubility promoters.

Fillers which can be added are, for example, inorganic fillers, such as oxides of magnesium, aluminum, silicon, titanium carbonate or calcium carbonate, calcium phosphate or magnesium phosphate, or organic fillers, such as lactose, sucrose, sorbitol, mannitol.

Suitable plasticizers are, for example, triacetin, triethyl citrate, glycerol monostearate, low molecular weight polyethylene glycols, or poloxamers.

Suitable solubility promoters are interface-active substances with an HLB value (Hydrophilic Lipophilic Balance) greater than 11, for example hydrogenated castor oil ethoxylated with 40 ethylene oxide units (Cremophor® RH 40), castor oil ethoxylated with 35 ethylene oxide units (Cremophor eL), Polysorbate 80, poloxamers or sodium lauryl sulfate.

Lubricants which can be used are stearates of aluminum, calcium, magnesium and tin, and also magnesium silicate, silicones and the like.

Flow agents which can be used are, for example, talc or colloidal silicon dioxide.

A suitable binder is, for example, microcrystalline cellulose.

Disintegrants may be crosslinked polyvinylpyrrolidone or crosslinked sodium carboxymethyl starch. Stabilizers may be ascorbic acid or tocopherol.

Dyes are e.g. iron oxides, titanium dioxide, triphenylmethane dyes, azo dyes, quinoline dyes, indigo tin dyes, carotenoids, in order to color the administration forms, opaquing agents such as titanium diodide or talc, in order to increase the light permeability and to save dyes.

The polymeric salts of active ingredients according to the invention can be granulated in an excellent manner and compressed to give tablets which, on account of the high solubility in aqueous media, exhibit extremely rapid active ingredient release. The improved solubility results in considerably improved bioavailability. The solubility is usually 0.05 to 5% (parts by weight of medicament/parts by weight of water). In addition, the bioavailability is very reproducible, i.e. there are relatively slight interindividual fluctuations.

The salts according to the invention can be formulated to give many different administration forms such as, for example, tablets, capsules, granules, powders, drug delivery systems, solutions, suppositories, transdermal systems, creams, gels, lotions, injection solutions, drops, juices, syrups.

### EXAMPLES

### Polymer A

Polyacrylic acid (MW 4000; K value 11)

### Polymer B

Maleic acid-acrylic acid (weight ratio 50/50) copolymer (MW 3000; K value 10)

### Polymer C

Methacrylic acid-acrylic acid (weight ratio 70/30) copolymer (MW 20 000; K value 12)

### Polymer D

Polyacrylic acid (MW 100 000; K value 38.6)

### Polymer E

Acrylic acid/2-sulfoethyl methacrylate weight ratio 90/10, K value 82

### Polymer F

Acrylic acid/itaconic acid weight ratio 90/10, K value 55

### Polymer G

Acrylic acid/2-acrylamido-2-methylpropanoic acid/acrylamide weight ratio 80/10/10, K value 74

### Polymer H

Acrylic acid/2-acrylamido-2-methylpropanoic acid weight ratio 98.2/10.8, K value 82

All of the K values were determined on 1% strength by weight aqueous solutions.

The disintegration of the tablets was carried out using an Erweka disintegration tester. The release was determined in accordance with Pharm. Eur. 6.0.

The crospovidone used was Kollidon CL-F, BASF AG, average particle size 30 μm.

The twin-screw extruder used for the preparation of extrudates was an extruder of the type ZSK 25 from Werner & Pfleiderer with a screw diameter of 16 mm and a length of 25D. The temperature of the variably heatable cylinders was set constant except for the chilled inlet cylinder depending on the product. The dies used had a diameter of 2 or 3 mm.

The water content of the extrudates was less than 3% by weight.

Determination of the amorphous state was carried out by means of polarization microscopy.

General remarks relating to the preparation procedure for polymers E-G

The polymerization was carried out in a 2 l reactor with anchor stirrer. The resulting polymer solutions were converted to powder by freeze-drying.

Demin. water: demineralized water

### Preparation of Polymer E

Copolymer of 90% by weight of acrylic acid and
10% by weight of 2-sulfoethyl methacrylate
Monomer amount: 240.0 g batch size: 802.8 g

| Initial charge total: 320.0 g | |
|---|---|
| 320.0 g | demin. water |
| Feed 1 total: 276.0 g | |
| 36.0 g | demin. water |
| 24.0 g | 2-sulfoethyl methacrylate |
| 216.0 g | acrylic acid |
| Feed 2 total: 124.4 g | |
| 122.0 g | demin. water |
| 2.4 g | sodium hypophosphite monohydrate |
| Feed 3 total: 82.4 g | |
| 2.4 g | sodium peroxodisulfate |
| 80.0 g | demin. water |

The initial charge was heated to an internal temperature of 90° C. at 120 rpm under nitrogen blanketing.

When an internal temperature of 90° C. had been reached, feeds 1 and 2 were metered in over 4 hours and feed 3 was metered in over 4 hours 15 minutes. At the end of feed 3, the mixture was left to afterpolymerize for a further 1 hour at 90° C. The mixture was then cooled to room temperature. This gave a slightly viscous, clear, yellow solution with a solids content of 29.9% by weight and a K value of 46.

### Preparation of Polymer F

Copolymer of 90% by weight of acrylic acid and 10% by weight
of itaconic acid
Monomer amount: 560.0 g batch size: 1651.2 g

| | |
|---|---|
| Allocation amount | feed material |

Initial charge total: 682.4 g

| | |
|---|---|
| 56.0 g | itaconic acid |
| 0.6 g | iron (II) sulfate heptahydrate 1.0% strength in water |
| 611.8 g | demin. water |
| 14.0 g | of feed 2 |

Feed 1 total: 702.8 g

| | |
|---|---|
| 504.0 g | acrylic acid |
| 196.0 g | demin. water |
| 2.8 g | sodium hypophosphite monohydrate |

Feed 2 total: 280.0 g

| | |
|---|---|
| 56.0 g | hydrogen peroxide 30.0% strength |
| 224.0 g | demin. water |

The initial charge was heated to an internal temperature of 95° C. at 120 rpm under nitrogen. When an internal temperature of 95° C. had been reached, feed 1 was metered in over 5 hours and feed 3 was metered in over 6 hours. At the end of feed 2, the mixture afterpolymerizes for a further 2 h at 95° C. The mixture was then cooled to room temperature. This gave a clear, colorless, slightly viscous solution with a solids content of 35.0% by weight and a K value of 55.

### Preparation of Polymer G

Copolymer of 80% by weight of acrylic acid,
10% by weight of 2-acrylamido-2-methylpropanesulfonic acid
and 10% by weight of acrylamide
Monomer amount: 240.0 g batch size: 800.4 g

| Amount | | feed material |
|---|---|---|
| | Initial charge total: 296.0 g | |
| | 296.0 g | demin. water |
| | Feed 1 total: 240.0 g | |
| | 192.0 g | acrylic acid |
| | 24.0 g | 2-acrylamido-2-methylpropanesulfonic acid |
| | 24.0 g | demin. water |
| | Feed 2 total: 182.0 g | |
| | 158.0 g | demin. water |
| | 24.0 g | acrylamide |
| | Feed 3 total: 82.4 g | |
| | 2.4 g | sodium peroxodisulfate |
| | 80.0 g | demin. water |

The initial charge was heated to an internal temperature of 90° C. at 120 rpm under nitrogen. When an internal temperature of 90° C. had been reached, feeds 1 and 2 were metered in over 4 hours and feed 3 was metered in over 4 hours 15 minutes. At the end of feed 3, the mixture was left to afterpolymerize for a further 1 h at 90° C. The mixture was then cooled to room temperature. This gave a clear, viscous solution with a solids content of 30.8% by weight and a K value of 74.

### Preparation of Polymer H

Copolymer of 89.2% by weight of acrylic acid and
10.8% by weight of 2-acrylamido-2-methylpropanesulfonic acid
Monomer amount: 242.1 g batch size: 802.5 g

| Amount | | feed material |
|---|---|---|
| | Initial charge total: 320.0 g | |
| | 320.0 g | demin. water |
| | Feed 1 total: 400.1 g | |
| | 158.0 g | demin. water |
| | 26.1 g | 2-acrylamido-2-methylpropanesulfonic acid |
| | 216.0 g | acrylic acid |
| | Feed 2 total: 82.4 g | |
| | 2.4 g | sodium peroxodisulfate |
| | 80.0 g | demin. water |

The initial charge was stirred at 120 rpm and heated to an internal temperature of 90° C. under nitrogen. When an internal temperature of 90° C. has been reached, feed 1 was metered in over 4 hours and feed 2 was metered in over 4 hours 15 minutes. At the end of feed 2, the mixture was left to afterpolymerize for a further 1 hour at 90° C. The mixture was then cooled to room temperature. This gave a clear, viscous, colorless solution with a solids content of 32.2% by weight and a K value of 82.

### Preparation of Polymeric Salts

### Example 1

### Solubilities of Loperamide in Aqueous Polymer Solutions

1.0 g of loperamide (free base) was added to 100.0 ml of aqueous polymer solution and stirred for 72 h. During this, the polymeric salt was formed up to its saturation solubility in water. The undissolved amount of loperamide was filtered off. The content of loperamide in the filtrate was determined by HPLC.

For comparison, the saturation solubility of loperamide was determined in demineralized water, as well as in hydrochloric acid. In the case of the latter, the corresponding loperamide hydrochloride is formed.

| | | Concentration of | |
|---|---|---|---|
| | | dissolved | |
| Polymer concentration | | loperamide | pH of the filtered |
| in water [% by wt.] | Polymer | [mg/ml] | solution |
| 0 | Water | <0.0170 | |
| 10 | A | 6.2 | 2.5 |
| 20 | A | 7.6 | |
| 10 | B | 3.9 | 2.0 |
| 20 | D | 5.9 | 1.9 |
| Hydrochloric acid 10% | — | <0.0149 | |

The solubility of loperamide in 20% strength solution of polymer A is improved compared with water at least by a factor of 447 and, compared with 10% strength by weight aqueous hydrochloric acid solution, by a factor of 510.

### Example 2

### Solubilities of Haloperidol in Aqueous Polymer Solutions

1.0 g of haloperidol (free base) was added to 100.0 ml of aqueous polymer solution and stirred for 72 h. During this, the polymeric salt is formed up to its saturation solubility in water. The undissolved amount of haloperidol was filtered off. The content of haloperidol in the filtrate was determined by HPLC.

For comparison, the saturation solubility of haloperidol was determined in demineralized water, as well as in hydrochloric acid. In the latter case, the corresponding haloperidol hydrochloride is formed.

To demonstrate that the solubility improvement is actually based on a salt formation and not on a solubilizing effect, a 20% solution of polymer A was adjusted to pH 10 with NaOH and the saturation solubility was determined therewith.

| | | Concentration | |
|---|---|---|---|
| | | of dissolved | pH of |
| Polymer concentration | | haloperidol | the filtered |
| in water [% by wt.] | Polymer | [mg/ml] | solution |
| 0 | Water | <0.0178 | 7.5 |
| 20 | A | 10.3 | 3.0 |
| 20 | C | 7.7 | 3.3 |
| 10 | D | 5.2 | 3.2 |
| 20 | D | 15.1 | 3.0 |
| 20 | Solution plus | <0.074 | 10.1 |
| | NaOH to pH 10 | | |
| Hydrochloric acid 10% | — | <0.0160 | |

The solubility of haloperidol in 20% strength polymer A solution is improved, compared with water, at least by a factor of 579 and, compared with a 10% strength by weight aqueous hydrochloric acid solution, by a factor of 644.

### Example 3

### Solubilities of Cinnarizine in Aqueous Polymer Solutions

1.0 g of cinnarizine (free base) was added to 100.0 ml of aqueous polymer solution and stirred for 72 h. During this, the polymeric salt was formed up to its saturation solubility in water. The undissolved amount of cinnarizine was filtered off. The content of cinnarizine in the filtrate was determined by HPLC.

For comparison, the saturation solubility of cinnarizine was determined in demineralized water, as well as in hydrochloric acid. In the latter case, the corresponding cinnarizine hydrochloride is formed.

| | | Concentration of |
|---|---|---|
| | | dissolved |
| Polymer concentration | | cinnarizine |
| in water [% by wt.] | Polymer | [mg/ml] |
| 0 | Water | 0.01 |
| 10 | A | 2.8 |
| 20 | A | 9.6 |
| 10 | C | 4.0 |
| 20 | C | 10.1 |
| 10 | D | 4.1 |
| 20 | D | 10.5 |
| Hydrochloric acid 10% | — | <0.79 |

The solubility of cinnarizine in 20% strength polymer A solution is improved, compared with water, at least by a factor of 480 and, compared with a 10% strength by weight aqueous hydrochloric acid solution, by a factor of 13.

### Example 4

### Preparation of a Salt of Loperamide and Polymer a by Spray-Drying

36 g of loperamide were dispersed in 1464 g of a 20% strength aqueous polymer A solution with stirring for 2 h. During this, some of the active ingredient already passed into solution. Shortly before the spray-drying, this dispersion was briefly heated to 90° C., during which the loperamide dissolved completely. Directly afterwards, the spray-drying was carried out under the following conditions: Entry gas temperature: 200° C.

Exit gas temperature: 103° C.
Spraying rate: 9.5 g/min
A dry powder was obtained.

### Product Properties:

Residual moisture: 2.1% (g/g)
Solubility of the polymeric loperamide salt in water measured as dissolved loperamide: 17.0 mg/ml

### Example 5

### Preparation of a Salt of Loperamide and Polymer B by Spray-Drying

36 g of loperamide were dispersed in 1464 g of a 20% strength aqueous polymer B solution with stirring for 2 h. During this, some of the active ingredient already passed into solution. Shortly prior to the spray-drying, this dispersion was heated briefly to 90° C., during which the loperamide dissolved completely. Directly afterwards, the spray-drying was carried out under the following conditions: Entry gas temperature: 168° C.

Exit gas temperature: 77° C.
Spraying rate: 9.5 g/min
A dry powder was obtained.

### Product Properties:

Residual moisture: 6.1% (g/g)
Solubility of the polymeric loperamide salt in water measured as dissolved loperamide: 13.3 mg/ml

### Example 6

### Preparation of a Salt of Haloperidol and Polymer a by Spray-Drying

54 g of haloperidol were dispersed in 1576 g of a 20% strength aqueous polymer A solution with stirring for 2 h. During this, some of the active ingredient already passed into solution. Shortly prior to the spray-drying, this dispersion was briefly heated to 90° C., during which the haloperidol dissolved completely. Directly afterwards, the spray-drying was carried out under the following conditions: Entry gas temperature: 193° C.

Exit gas temperature: 85° C.
Spraying rate: 12.5 g/min
A dry powder was obtained.

### Product Properties:

Residual moisture: 2.0% (g/g)
Solubility of the polymeric haloperidol salt in water measured as dissolved haloperidol: 34.1 mg/ml

### Example 7

### Preparation of a Salt of Haloperidol and Polymer B Solution by Spray-Drying

58 g of haloperidol were dispersed in 1692 g of a 20% strength aqueous polymer B solution with stirring for 2 h. During this, some of the active ingredient already passed into solution. Shortly prior to the spray-drying, this dispersion was briefly heated to 90° C., during which the haloperidol dissolved completely. Directly afterwards, the spray-drying was carried out under the following conditions: Entry gas temperature: 173° C.

Exit gas temperature: 91° C.
Spraying rate: 8.7 g/min
A dry powder was obtained.

### Product Properties:

Residual moisture: 5.7% (g/g)
Solubility of the polymeric haloperidol salt in water measured as dissolved haloperidol: 17.3 mg/ml

### Example 8

### Preparation of a Salt of Cinnarizine and Polymer a by Spray-Drying

24.5 g of cinnarizine were dispersed in 1725.5 g of a 20% strength aqueous polymer A solution with stirring for 2 h. During this, some of the active ingredient already passed into solution. Shortly prior to the spray-drying, this dispersion was briefly heated to 90° C., during which the cinnarizine dissolved completely. Directly afterwards, the spray-drying was carried out under the following conditions: Entry gas temperature: 177° C.

Exit gas temperature: 95° C.
Spraying rate 17.6 g/min
A dry powder was obtained.

### Product Properties:

Residual moisture: 2.1% (g/g)
Solubility of the polymeric cinnarizine salt in water measured as dissolved cinnarizine: 7.9 mg/ml

### Example 9

### Preparation of a Salt of Loperamide and Polymer a by Dissolution in an Organic Solvent and Subsequent Evaporation

5 g of loperamide were dissolved in 150 g of a 30% strength solution of polymer A in ethanol with stirring for 2 h. This solution was evaporated to dryness on a rotary evaporator. The resulting solid was then ground to give a powder.

### Product Properties:

Residual moisture: 1.4% (g/g)
Solubility of the polymeric loperamide salt in water measured as dissolved loperamide: 15.9 mg/ml

### Example 10

### Preparation of a Salt of Haloperidol and Polymer a by Dissolution in an Organic Solvent and Subsequent Evaporation

10 g of haloperidol were dissolved in 150 g of a 30% strength solution of polymer A in ethanol with stirring for 2 h. This solution was evaporated to dryness on a rotary evaporator at 80° C. The resulting solid was then ground to give a powder.

### Product Properties:

Residual moisture: 1.6% (g/g)
Solubility of the polymeric haloperidol salt in water measured as dissolved haloperidol: 25.4 mg/ml

### Example 11

### Preparation of a Salt of Loperamide and Polymer a by Extrusion

500 g of loperamide and 4500 g of polymer A were firstly homogeneously mixed in a mixer and then extruded in a twin-screw extruder at 130° C. The residence time was 3.5 min. The resulting strands with a thickness of 2 mm were then ground to give a powder.

### Product Properties:

Residual moisture: 1.4% (g/g)
Solubility of the polymeric loperamide salt in water measured as dissolved loperamide: 16.2 mg/ml

### Example 12

### Preparation of a Salt of Cinnarizine and Polymer a by Extrusion

700 g of cinnarizine and 4500 g of polymer A were firstly homogeneously mixed in a mixer and then extruded in a twin-screw extruder at 135° C. The residence time was 3.5 min. The resulting strands with a thickness of 2 mm were then ground to give a powder.

### Product Properties:

Residual moisture: 1.6% (g/g)
Solubility of the polymeric cinnarizine salt in water measured as dissolved cinnarizine: 7.2 mg/ml

### Example 13

### Preparation of a Salt of Famotidine and Polymer G by Extrusion

500 g of famotidine, 3500 g of polymer 3 and 800 g of poloxamer 188 were firstly homogeneously mixed in a mixer and then extruded in a twin-screw extruder at 140° C. The residence time was 4 min. The resulting strands of thickness 3 mm were then ground to give a powder.

### Product Properties:

Residual moisture: 0.5% (g/g)
The ground powder was amorphous. Solubility of the polymer famotidine salt in water measured as dissolved famotidine: 45.3 mg/ml

### Example 14

### Preparation of a Salt of Famotidine and Polymer F by Extrusion

500 g of famotidine and 4500 g of polymer F were firstly homogeneously mixed in a mixer and then extruded in a twin-screw extruder at 130° C. 750 g of water were fed in via a second metering point. The residence time was 2 min. The resulting strands of thickness 3 mm were granulated, dried and then ground to give a powder.

### Product Properties:

Residual moisture: 2.5% (g/g)
The ground powder was amorphous. Solubility of the polymeric famotidine salt in water measured as dissolved famotidine: 22.5 mg/ml

### Example 15

### Preparation of a Salt of Cinnarizine and Polymer H by Extrusion

700 g of cinnarizine, 3000 g of polymer H and 1000 g of PEG 1500 were firstly homogeneously mixed in a mixer and then extruded in a twin-screw extruder at 140° C. The residence time was 2 min. The resulting strands of thickness 2 mm were then ground to give a powder.

### Product Properties:

Residual moisture: 0.5% (g/g)
The ground powder was amorphous. Solubility of the polymeric cinnarizine salt in water measured as dissolved cinnarizine: 9.3 mg/ml

### Example 16

### Preparation of a Salt of Cinnarizine and Polymer E by Extrusion

600 g of cinnarizine and 4500 g of polymer E were simultaneously metered into the twin-screw extruder using two powder meterings in the ratio 1:10. 650 g of ethanol were fed in via a further metering point. Extrusion was carried out at 125° C., during which the residence time was 2 min. The resulting strands of thickness 2 mm were then ground to give a powder.

### Product Properties:

Residual moisture: 1.7% (g/g)
The ground powder was amorphous. Solubility of the polymeric cinnarizine salt in water measured as dissolved cinnarizine: 5.7 mg/ml

### Example 17

### Preparation of a Salt of Cinnarizine and Polymer E by Extrusion

600 g of cinnarizine and 4000 g of polymer E were firstly homogeneously mixed in a mixer and then fed into a twin-screw extruder. 800 g of propylene glycol were fed in via a further metering point. Extrusion was carried out at 115° C., during which the residence time was 2 min. The resulting strands of thickness 2 mm were then ground to give a powder.

### Product Properties:

Residual moisture: 1.4% (g/g)
The ground powder was amorphous. Solubility of the polymeric cinnarizine salt in water measured as dissolved cinnarizine: 8.0 mg/ml

### Example 18

### Preparation of Tablets with a Polymeric Salt of Loperamide and Polymer A

17 g of polymeric loperamide salt of Example 11 were mixed with 150 g of microcrystalline cellulose, 118 g of dicalcium phosphate, 12 g of crospovidone and 3 g of magnesium stearate and compressed on an eccentric tableting press to give tablets with the following properties.

### Diameter: 10 mm

### Weight: 300 mg

Fracture resistance: 60 N

### Disintegration: 33 s

Active ingredient release in water: 99% after 15 min

### Example 19

### Preparation of Tablets with a Polymeric Salt of Haloperidol and Polymer A

65 g of polymeric haloperidol salt of Example 4 were mixed with 110 g of microcrystalline cellulose, 110 g of dicalcium phosphate, 12 g of crospovidone and 3 g of magnesium stearate and compressed on a rotary tableting machine to give tablets with the following properties.

### Diameter: 10 mm

### Weight: 300 mg

Fracture resistance: 80 N

### Disintegration: 68 s

Active ingredient release in water: 99% after 15 min

1.-21. (canceled)

22. A polymeric water-soluble salt of medicaments that are sparingly soluble in water, consisting of a polymer with anionic character that is soluble in water at pH values of 2-13 and a sparingly soluble medicament with cation character.

23. The polymeric salt according to claim 22, wherein the polymer with anion character is a fraction of carboxyl-group-carrying monomers at least 40% by weight.

24. The polymeric salt according to claim 22, wherein the polymer with anion character is not gel-forming.

25. The polymeric salt according to claim 22, wherein the medicament has at least one and at most two groups capable of salt formation.

26. The polymeric salt according to claim 22, wherein the polymeric medicament salt has a higher solubility in water than the medicament and the corresponding hydrochloride of the medicament.

27. The polymeric salt according to claim 22, wherein the polymer with anion character in 1% strength by weight aqueous solution has a K value in accordance with Fikentscher of less than 150.

28. The polymeric salt according to claim 22, wherein the polymer with anion character in 1% strength by weight aqueous solution has a K value in accordance with Fikentscher of less than 90.

29. The polymeric salt according to claim 22, wherein the polymer with anion character in 1% strength by weight aqueous solution has a K value in accordance with Fikentscher of less than 50.

30. The polymeric salt according to claim 22, wherein the polymer with anion character can comprise, besides carboxyl groups, phosphoric acid or sulfonic acid groups or a combination of these.

31. The polymeric salt according to claim 22, wherein the polymer with anion character comprises, as carboxyl-group-carrying monomers, acrylic acid, methacrylic acid or maleic acid or mixtures thereof.

32. The polymeric salt according to claim 22, comprising, as carboxyl-group-carrying monomers, a combination of itaconic acid with acrylic acid, methacrylic acid or maleic acid.

33. The polymeric salt according to claim 22, comprising 1 to 20% by weight of monomers carrying sulfonic acid groups.

34. The polymeric salt according to claim 22, comprising 1 to 20% by weight of neutral comonomers.

35. The polymeric salt according to claim 22, wherein a polymer is used whose K value in 1% strength by weight aqueous solution is less than 90.

36. The polymeric salt according to claim 22, wherein a polymer is used whose K value in 1% strength by weight aqueous solution is less than 50.

37. A solid administration form comprising the polymeric salts according to claim 22.

38. The solid administration form according to claim 37, produced by compression.

39. A method for producing polymeric, water-soluble salts of medicaments according to claim 22, wherein a polymer with anion character that is water-soluble at pH values of 2-13 and a sparingly soluble medicament with cation character are dissolved in a solvent and the polymeric medicament salt is isolated from the solution.

40. The method according to claim 39, wherein the polymeric medicament salt is isolated by drying the solution.

41. The method according to claim 39, wherein the polymeric medicament salt is obtained from the solution by precipitation as a result of cooling or an addition of a nonsolvent.

42. A method for producing polymeric, water-soluble salts of medicaments according to claim 22, wherein the preparation takes place by combined processing of the polymers with anion character and active ingredient in an extruder.
